(19) United States
(12) Patent Application Publication
(10) Pub. No.: US 2020/0223927 A1
(43) Pub. Date: Jul. 16, 2020

(54) METHODS AND COMPOSITIONS FOR TREATING MULTIPLE SCLEROSIS

(71) Applicants: Avner YAYON, Moshav Sitria (IL); Eran ROM, Rehovot (IL)

(72) Inventors: Avner YAYON, Moshav Sitria (IL); Eran ROM, Rehovot (IL)

(21) Appl. No.: 16/650,476

(22) PCT Filed: Sep. 27, 2018
(86) PCT No.: PCT/IL2018/051072

§ 371 Date: Mar. 25, 2020

Related U.S. Application Data
(60) Provisional application No. 62/564,277, filed on Sep. 28, 2017.

(51) Int. Cl. C07K 16/28; A61P 25/00

(52) U.S. Cl. CPC C07K 16/2863; A61K 2039/505; A61P 25/00

(57) ABSTRACT

The present invention relates to compositions and methods of treating multiple sclerosis. Particularly, the present invention relates to pharmaceutical compositions comprising antibodies having affinity to both fibroblast growth factor receptor 2 (FGFR2) and FGFR3 for use in treating multiple sclerosis.

### FIELD OF THE INVENTION

The present invention relates to compositions and methods of treating multiple sclerosis. Particularly, the present invention relates to pharmaceutical compositions comprising antibodies having affinity to both fibroblast growth factor receptor 2 (FGFR2) and FGFR3 for use in treating multiple sclerosis.

### BACKGROUND OF THE INVENTION

### Fibroblast Growth Factors and their Receptors

Fibroblast Growth Factor ligands (FGFs) constitute a family of at least eighteen functionally and structurally related polypeptides that are developmentally regulated and expressed in a wide variety of tissues and diseases. FGFs are widely known as cell mitogens and as stimulators of cell migration and differentiation. In addition, FGFs have been shown to play a major role in various physiological and pathophysiological conditions, such as in skeletal and limb development, wound healing, tissue repair, hematopoiesis, angiogenesis, and tumorigenesis. All members of the FGF family share a homology core domain of about 120 amino acids, 28 amino acid residues are highly conserved and four are identical. The adjacent N- and C-termini are of variable length and share limited homology. The core domain comprises both the primary receptor binding sites and a heparan-binding domain which are distinct from each other.

The biological response of cells to FGF is mediated through specific, high affinity (Kd 20-500 pM) cell surface receptors that possess intrinsic tyrosine kinase activity and are phosphorylated upon binding of FGF. Five distinct Fibroblast Growth Factor Receptors (FGFRs) have been identified, FGFR1-4 are transmembrane-protein kinases while FGFRS lacks a tyrosine kinase domain. The FGFR extracellular domain consists of three immunoglobulin-like (Ig-like) domains (D1, D2 and D3), a heparan binding domain and an acidic box. Alternative splicing of D3 in FGFR1, FGFR2 and FGFR3 mRNAs generates distinct receptor isoforms (designated as FGFR1IIIb, FGFR1IIIc, FGFR2IIIb, FGFR2IIIc, FGFR3IIIb, and FGFR3IIIc) which together with FGFR4 constitute a family of seven different receptor subtypes, having unique ligand specificity and tissue distribution pattern.

Because of the key role FGFs display in various pathophysiological diseases, these ligands and their receptors serve as potential targets in treating diseases or conditions associated with the FGF signaling pathway.

International Patent Publication WO 2007/144893, to some of the inventors of the present invention, discloses antibodies having cross-reactivity to FGFR2 and/or FGFR3 which are useful in the treatment of cell proliferative diseases of epithelial origin.

International Patent Publication WO 2008/038287, to some of the inventor of the present invention, discloses FGF2 and FGF4 polypeptides comprising a truncated N-terminus and having increased receptor specificity. The polypeptides are useful in treating skeletal and coronary and peripheral vascular disorders.

### Multiple Sclerosis

Multiple sclerosis (MS) is an autoimmune neurodegenerative disease. It is a severe and chronic disabling disease, the symptoms of which include pain and tingling in the arms and legs; localized and generalized numbness, muscle spasm and weakness; difficulty with balance when standing or walking; difficulty with speech and swallowing; cognitive deficits; fatigue; and bowel and bladder dysfunction.

The underlying cause of multiple sclerosis (MS) is irreversible axonal injury and loss. This axonal pathology is attributed to detrimental effects of inflammatory mediators on mitochondrial function that are exacerbated by concurrent demyelination. The loss of myelin not only increases axonal susceptibility to inflammatory mediators, but disrupts metabolic support provided by the myelinating oligodendrocytes; a combination of effects that results in profound axonal energy deficits, and ultimately, in irreversible axonal damage. In experimental models of immune-mediated demyelination these effects on axonal health are mitigated by remyelination carried out by oligodendrocytes derived from NG2⁺ Oligodendrocyte Progenitor Cells (OPC). However this endogenous repair mechanism fails in over 80% of MS lesions, leaving affected axons increasingly vulnerable to inflammatory and metabolic stress.

The reason why the endogenous neuroprotective response fails in MS is unclear, but the continuing presence of OPC in many MS lesions suggests the involvement of various factors which inhibit OPC differentiation into myelination-competent oligodendrocytes. OPC numbers are lowest, and in some cases undetectable, in chronically demyelinated lesions, but in early active lesions their density may exceed that in adjacent normal appearing white matter. These lesions also contain Olig2^{bright}NogoA⁺ cells thought to represent transitioning or more differentiated oligodendrocytes which are derived from the OPC population. The local microenvironment can therefore support OPC recruitment, survival and differentiation at this stage of lesion development.

FGF2 was shown to be over-expressed within lesions of multiple sclerosis. In vitro, FGF2 was found to support the proliferation and migration of OPCs, but to inhibit the differentiation of OPC to oligodendrocytes (Bansal, R. Dev. Neurosci. 2002, 24(1): 35-46). FGF2 was also shown to be a negative regulator of remyelination (Butt and Dinsdale, J. Neuroimmunol. 2005, 166(1): 75-87; Azim et al., Glia, 2012, 60(12)1977-1990). Yet, FGF2 is also neuroprotective following ischemic, neurodegenerative and neuroinflammatory insults to the central nervous system (CNS), an effect already exploited to reduce disease burden in experimental autoimmune encephalomyelitis (EAE), an animal model for MS (Alzheimer and Werner, Adv. Exp. Med. Biol., 2002, 513: 335-351; Yemisci et al., J. Cereb. Blood Flow Metab. 2015, 35(3): 469-475; Rottlander et al., Immunol., 2011, 133(3): 370-378).

FGF2-mediated inhibition of myelination was investigated under in vitro conditions and was shown to be FGFR2 mediated (Lindner et al., Multiple Sclerosis J., 2013, 19: (S1) 565).

There remains an unmet need for improved compositions and methods for treating multiple sclerosis.

### SUMMARY OF THE INVENTION

The present invention provides compositions comprising antibodies or antigen-binding portions thereof having affinity to fibroblast growth factor receptor 2 (FGFR2), preferably having affinity to both FGFR2 and FGFR3, for use in treating, slowing the progression of, or ameliorating one or more symptoms of multiple sclerosis in a subject diagnosed with the disease.

It is now disclosed for the first time that when antibodies having high affinity to both FGFR2 and FGFR3 were injected to mice having experimental autoimmune encephalomyelitis (EAE), the antibodies reduced the severity of the disease as determined by clinical evaluation of the mice, and even prolonged their survival. Histological examination of the brain sections from the treated mice indicated that the antibodies significantly reduced the demyelination in the brains of the treated mice.

The present invention therefore provides an efficient means for treating multiple sclerosis.

According to one aspect, the present invention provides a method of treating, slowing the progression of, or ameliorating one or more symptoms of multiple sclerosis, the method comprising administering to a subject in need of such treatment a therapeutically effective amount of an antibody or an antigen-binding portion thereof having affinity to fibroblast growth factor receptor 2 (FGFR2), optionally with cross-reactivity to other FGFR.

According to some embodiments, the antibody or the antigen-binding portion thereof being administered to the subject has affinity to both FGFR2 and FGFR3. According to additional embodiments, the antibody or the antigen-binding portion thereof binds both FGFR2 and FGFR3 with affinity of at least 50 nM. According to further embodiments, the antibody or the antigen-binding portion thereof binds both FGFR2 and FGFR3 with affinity of at least 10 nM. According to yet further embodiments, the antibody or the antigen-binding portion thereof is substantially devoid of affinity to FGFR1.

According to additional embodiments, the antibody or the antigen-binding portion thereof being administered to the subject is selected from the group consisting of a monoclonal antibody, a proteolytic fragment of the monoclonal antibody, and a single chain antibody.

According to some embodiments, the antibody or the antigen-binding portion thereof comprises a V_{H}-CDR3 region having an amino acid sequence as set forth in SEQ ID NO:1 and a V_{L}-CDR3 region having an amino acid sequence as set forth in SEQ ID NO:2.

According to one embodiment, the proteolytic fragment of the antibody is single chain Fv. According to a certain embodiment, the single chain Fv has an amino acid sequence as set forth in SEQ ID NO:5.

According to additional embodiments, administering the pharmaceutical composition is performed by intravenous, intraarterial, intramuscular, or intraperitoneal injection or infusion.

According to further embodiments, administering the pharmaceutical composition is performed by intravenous, intraarterial, intramuscular, or intraperitoneal injection or infusion once daily for 1, 2, 3, 4, 5, 6, or more consecutive days, or until the symptoms of the disease are eliminated.

According to yet further embodiments, administering the pharmaceutical composition is performed by intravenous, intraarterial, intramuscular, or intraperitoneal injection or infusion every other day for 2, 3, 4, 5, or more times, or until the symptoms of the disease are eliminated.

According to some embodiments, the method further comprises a step of administering an additional pharmaceutical composition which comprises a therapeutically effective amount of an agent known to treat, slow the progression of, or ameliorate one or more symptoms of multiple sclerosis.

According to further embodiments, the agent known to treat multiple sclerosis is selected from the group consisting of ocrelizumab, beta interferon, glatiramer acetate, dimethyl fumarate, and fingolimod.

According to yet further embodiments, administering the additional pharmaceutical composition is performed by oral or parenteral administration route.

According to still further embodiments, the parenteral administration route is selected from the group consisting of intravenous, intraarterial, intramuscular, and intraperitoneal injection or infusion.

According to further embodiments, administering the pharmaceutical composition which comprises the antibody or antigen-binding portion thereof having affinity to both FGFR2 and FGFR3 and the pharmaceutical composition comprising the agent known to treat multiple sclerosis is performed concomitantly or subsequently to each other.

According to another aspect, the present invention provides a pharmaceutical composition comprising an antibody or an antigen-binding portion thereof having affinity to FGFR2, optionally with cross-reactivity to other FGFR, for use in treating, slowing the progression of, or ameliorating one or more symptoms of multiple sclerosis according to the principles of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 depicts the average mouse body weight of EAE untreated mice and of EAE mice treated with the anti-FGF2/3 antibodies PRO-007. The average body weight of the following groups was measured: EAE mice treated with PRO-007 at a dose of 0.08 mg/kg every 48 hrs (Group A, bleu), EAE mice treated with PRO-007 at a dose of 0.08 mg/kg every 24 hrs (Group B, orange), EAE mice treated with PRO-007 at a dose of 1.2 mg/kg every 48 hrs (Group C, grey), and untreated mice (Group D, yellow).

FIG. 2 depicts the clinical scoring of EAE mice. The clinical scoring of the following groups was determined: EAE mice treated with PRO-007 at a dose of 0.08 mg/kg every 48 hrs (Group A, bleu), EAE mice treated with PRO-007 at a dose of 0.08 mg/kg every 24 hrs (Group B, orange), EAE mice treated with PRO-007 at a dose of 1.2 mg/kg every 48 hrs (Group C, grey), and untreated mice (Group D, yellow).

FIG. 3 depicts the clinical scoring of EAE mice at the end of the experiment. The clinical scoring of the following groups was determined: EAE mice treated with PRO-007 at a dose of 0.08 mg/kg every 48 hrs (Group A), EAE mice treated with PRO-007 at a dose of 0.08 mg/kg every 24 hrs (Group B), EAE mice treated with PRO-007 at a dose of 1.2 mg/kg every 48 hrs (Group C), and untreated mice (Group D). The difference between Group A and Group D was statistically different (*p<0.05).

FIG. 4 depicts the survival rate of the EAE mice. The survival rate of the following groups was determined: EAE mice treated with PRO-007 at a dose of 0.08 mg/kg every 48 hrs (Group A, bleu), EAE mice treated with PRO-007 at a dose of 0.08 mg/kg every 24 hrs (Group B, orange), EAE mice treated with PRO-007 at a dose of 1.2 mg/kg every 48 hrs (Group C, grey), and untreated mice (Group D, yellow).

FIG. 5 shows micrographs of brain sections from EAE mice stained with either luxor blew (LB) or with hematoxylin eosin (HE). Brain sections from untreated EAE mice or from mice treated with PRO-007 at a dose of 0.08 mg/kg every 48 hrs are shown.

FIG. 6 shows immunohistochemistry evaluation of myelin basic protein (MBP) in brain sections from EAE untreated mice and from EAE mice treated with PRO-007 at a dose of 0.08 mg/kg every 48 hrs.

FIG. 7 shows the percentage of healthy white matter in spinal cord samples from EAE untreated mice and from EAE mice treated with PRO-007 at a dose of 0.08 mg/kg every 48 hrs.

### DETAILED DESCRIPTION OF THE INVENTION

The present invention provides methods for treating, attenuating the progression of, or ameliorating one or more symptoms of multiple sclerosis, the methods comprise administering to a subject in need of such treatment a pharmaceutical composition comprising antibodies having high affinity to fibroblast growth factor receptor 2 (FGFR2), preferably having cross-reactivity to FGFR3.

Multiple sclerosis (MS) is an inflammatory disease of the CNS characterized by neurological impairment of variable extent, resulting from demyelination and axonal damage. This disease usually occurs in young adults, more commonly in women. The default response to demyelination in the CNS is remyelination by oligodendrocytes derived from an endogenous pool of Oligodendrocytes Progenitor Cells (OPC), but in MS this intrinsic repair mechanism frequently fails resulting in chronically demyelinated plaques of gliotic scar tissue.

### Definitions

The term “Fibroblast Growth Factor (FGF)” as used herein refers to a family of structurally related polypeptides, expressed in a wide variety of cells and tissues. Overall, the FGFs share between 17-72% amino acid sequence homology and a high level of structural similarity. A homology core of about 120 amino acids is highly conserved and has been identified in all members of the family.

The term “FGF2” as used herein, also known as basic FGF, bFGF, prostatin and heparan binding growth factor 2, is highly conserved among species and has been shown to stimulate the proliferation of a wide variety of cell types. The sequence of FGF2 has been disclosed in U.S. Pat. Nos. 4,994,559; 5,155,214; 5,439,818 and 5,604,293. The core domain of FGF2 extends from amino acid Lys30 to Lys154.

The term “FGFR” as used herein denotes a receptor specific for FGF molecule(s) which is necessary for transducing the signal exerted by FGF to the cell interior, typically comprising an extracellular ligand-binding domain, a single transmembrane helix, and a cytoplasmic domain that contains a tyrosine kinase activity. The FGFR extracellular domain consists of three immunoglobulin-like (Ig-like) domains (D1, D2 and D3), a heparin binding domain and an acidic box. Five FGFR genes that encode for multiple receptor protein variants are known. Alternative splicing of the FGFR2 mRNAs generates at least two known isoforms of the receptors, FGFR2IIIb and FGFR2IIIc.

The term “FGFR2 specific” as used herein refers to an agent, e.g., an antibody or a fragment thereof, that has higher affinity (K_{D}<50 nM) to FGFR2 polypeptide than to another FGF receptor protein. It is to be explicitly understood that the term “FGFR2 specific” does not exclude or preclude situations wherein the agent, e.g., an antibody or a fragment thereof, has some affinity or binding to another FGF receptor subtype. It is further to be understood that if the affinity of the agent to another receptor subtype is clinically important for the therapeutic utility observed, this is explicitly encompassed within the scope of the claimed invention.

The term “affinity” as used herein refers to the attraction between an antigen and an antibody which induces their binding. As used herein, by the term “antibody which has affinity for fibroblast growth factor receptor 2 (FGFR2) optionally with cross-reactivity to other FGF receptors” is meant that the antibody possesses high affinity to FGFR2 (KD<50 nM), but may also have affinity to other FGFRs. Preferably, the antibody has an affinity of at least 10 nM for both FGFR2 and FGFR3. In some embodiments, the affinity for FGFR2 and FGFR3 is approximately equal. In other embodiments, the particular antibody may have a greater affinity for one or the other of these receptors.

By the term “substantially devoid of affinity to FGFR1” is meant that the antibody or an antigen binding fragment thereof has low affinity to FGFR1 (KD>100 nM). A non-limiting example for an antibody which has high affinity for FGFR2 with cross-reactivity to other FGF receptors but is substantially devoid of affinity to FGFR1 is PRO-007 which has high affinity (KD<50 nM) to both FGFR2 and FGFR3 but low affinity to FGFR1.

The term “ligand-dependent receptor activation” as used herein refers to activation of the FGF receptor that is dependent on the amount of ligand presented to the receptor. The term “ligand-independent receptor activation” or “constitutive receptor activation” refers to activation of the FGF receptor that is independent of ligand presentation. Receptors are usually activated by their corresponding ligand in a dose dependent manner Certain mutations cause receptors to be constitutively activated independent of their ligand.

Many antibodies block only ligand-dependent receptor activation but not ligand-independent receptor activation. The antibodies of the present invention are unique in that they block both ligand-dependent and ligand-independent receptor activation.

One embodiment of the present invention is directed to molecules comprising an antigen binding domain which blocks both ligand-dependent and ligand-independent activation of FGFR2 and/or FGFR3.

### Antibodies

Antibodies, or immunoglobulins, comprise two heavy chains linked together by disulfide bonds and two light chains, each light chain being linked to a respective heavy chain by disulfide bonds in a “Y” shaped configuration. Proteolytic digestion of an antibody yields Fv (Fragment variable) and Fc (fragment crystalline) domains. The antigen binding domains, Fab, include regions where the polypeptide sequence varies. The term F(ab′)₂ represents two Fab′ arms linked together by disulfide bonds. The central axis of the antibody is termed the Fc fragment. Each heavy chain has at one end a variable domain (V_{H}) followed by a number of constant domains (C_{H}). Each light chain has a variable domain (V_{L}) at one end and a constant domain (C_{L}) at its other end, the light chain variable domain being aligned with the variable domain of the heavy chain and the light chain constant domain being aligned with the first constant domain of the heavy chain (CH1).

The variable domains of each pair of light and heavy chains form the antigen-binding site. The domains on the light and heavy chains have the same general structure and each domain comprises four framework regions, whose sequences are relatively conserved, joined by three hypervariable domains known as complementarity determining regions (CDR1-3). These domains contribute specificity and affinity of the antigen-binding site.

Additionally, complementarity determining region (CDR) grafting may be performed to alter certain properties of the antibody molecule including affinity or specificity. A non-limiting example of CDR grafting is disclosed in U.S. Pat. No. 5,225,539.

The isotype of the heavy chain (gamma, alpha, delta, epsilon or mu) determines immunoglobulin class (IgG, IgA, IgD, IgE or IgM, respectively). The light chain is either of two isotypes (kappa, κ or lambda, λ) found in all antibody classes.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)₂ fragments. Further included within the scope of the invention are chimeric antibodies; human and humanized antibodies; recombinant and engineered antibodies, and fragments thereof. Furthermore, the DNA encoding the variable region of the antibody can be inserted into the DNA encoding other antibodies to produce chimeric antibodies (see, for example, U.S. Pat. No. 4,816,567). Single chain antibodies fall within the scope of the present invention.

According to certain embodiments, the antigen-binding portion of an antibody which has affinity for FGFR2 with cross-reactivity to other FGF receptors, e.g., FGFR3, comprises a fragment of a monoclonal antibody such as the Fab, F(ab′)₂, or a single chain variable (scFv) fragments.

The term “single chain variable fragment (scFv)” as used herein is meant a fusion of the variable regions of the heavy and light chains of immunoglobulin, linked together with a short (usually serine, glycine) linker. Single chain antibodies can be single chain composite polypeptides having antigen binding capabilities and comprising amino acid sequences homologous or analogous to the variable regions of an immunoglobulin light and heavy chain (linked V_{H}-V_{L} or single chain Fv (scFv)). Both V_{H} and V_{L} may copy natural monoclonal antibody sequences or one or both of the chains may comprise a CDR-FR construct of the type described in U.S. Pat. No. 5,091,513, the entire contents of which are incorporated herein by reference. The separate polypeptides analogous to the variable regions of the light and heavy chains are held together by a polypeptide linker. Methods of production of such single chain antibodies, particularly where the DNA encoding the polypeptide structures of the V_{H} and V_{L} chains are known, may be performed in accordance with the methods described, for example, in U.S. Pat. Nos. 4,946,778, 5,091,513 and 5,096,815, the entire contents of each of which are incorporated herein by reference.

In certain embodiments, the present invention provides antibodies which block ligand-dependent and constitutive ligand-independent FGF receptor activation comprising a V_{H}-CDR3 region (complementarity determining region 3 of the heavy chain) having a polypeptide sequence as set forth in SEQ ID NO: 1 and a corresponding V_{L}-CDR3 region (complementarity determining region 3 of the light chain) having a polypeptide sequence as set forth in SEQ ID NO:2. The corresponding polynucleotide sequences of the V_{H}-CDR3 and V_{L}-CDR3 regions are set forth in SEQ ID NO: 3 and SEQ ID NO: 4, respectively. The amino acid and nucleotide sequences are as follows:

SEQ ID NO: 1
SYYPDFDY
SEQ ID NO: 2
QSYASQGIHY
SEQ ID NO: 3
TCTTATTATCCTGATTTTGATTAT
SEQ ID NO: 4
CAGTCTTATGCTTCTCAGGGTATTCATTAT

According to some embodiments, the antibody which blocks both ligand-dependent receptor activation and constitutive (ligand-independent) receptor activation has binding affinity of at least 50 nM to both FGFR2 and FGFR3. Preferably, the antibody has affinity of at least 10 nM for both FGFR2 and FGFR3. According to other embodiments, the antigen-binding portion of an isolated antibody having affinity for FGFR2 and/or FGFR3 is selected from monoclonal antibodies, a monoclonal antibody fragment or an antibody-fusion protein. The affinity of a given antibody to various receptor subtypes can be easily measured using methods known to one of skill in the art. By way of example, antibody affinities to receptor subtypes may conveniently be measured using the BIACORE® technology (AB Corporation, Sweden), among others. Using this technology, antibodies of the present invention were found to have affinities of less than 10 nM to both FGFR2 and FGFR3, whereas affinities to FGFR1 were undetectable using this method.

According to a certain embodiment, the antibody fragment is a single chain Fv molecule (scFv) having the amino acid sequence as set forth in SEQ ID NO:5. This scFv is designated throughout the specification PRO-007 characterized by having high binding affinity to both FGFR2 and FGFR3 but low affinity to FGFR1. The corresponding polynucleotide encoding the scFv of SEQ ID NO:5 has a nucleotide sequence as set forth in SEQ ID NO:6. The CDRs are bold and underlined.

SEQ ID NO: 5:
MVQLQQSGPGLVKPSQTLSLTCAISGDSVSSNSAAWNWIRQSPGRGLEWLG
RTYYRSKWYNDYAVSVKSRITINPDTSKNQFSLQLNSVTPEDTAVYYCARS
YYPDFDYWGQGTLVTVSSAGGGSGGGGSGGGGSGGGGSDIELTQPPSVSVA
PGQTARISCSGDALGDKYASWYQQKPGQAPVLVIYDDSDRPSGIPERFSGS
NSGNTATLTISGTQAEDEADYYCQSYASQGIHYVFGGGTKLTVLGQ
SEQ ID NO: 6:
ATGGTGCAATTGCAACAGTCTGGTCCGGGCCTGGTGAAACCGAGCCAAACC
CTGAGCCTGACCTGTGCGATTTCCGGAGATAGCGTGAGCAGCAACAGCGCG
GCGTGGAACTGGATTCGCCAGTCTCCTGGGCGTGGCCTCGAGTGGCTGGGC
CGTACCTATTATCGTAGCAAATGGTATAACGATTATGCGGTGAGCGTGAAA
AGCCGGATTACCATCAACCCGGATACTTCGAAAAACCAGTTTAGCCTGCAA
CTGAACAGCGTGACCCCGGAAGATACGGCCGTGTATTATTGCGCGCGTTCT
TATTATCCTGATTTTGATTATTGGGGCCAAGGCACCCTGGTGACGGTTAGC
TCAGCGGGTGGCGGTTCTGGCGGCGGTGGGAGCGGTGGCGGTGGTTCTGGC
GGTGGTGGTTCCGATATCGAACTGACCCAGCCGCCTTCAGTGAGCGTTGCA
CCAGGTCAGACCGCGCGTATCTCGTGTAGCGGCGATGCGCTGGGCGATAAA
TACGCGAGCTGGTACCAGCAGAAACCCGGGCAGGCGCCAGTTCTGGTGATT
TATGATGATTCTGACCGTCCCTCAGGCATCCCGGAACGCTTTAGCGGATCC
AACAGCGGCAACACCGCGACCCTGACCATTAGCGGCACTCAGGCGGAAGAC
GAAGCGGATTATTATTGCCAGTCTTATGCTTCTCAGGGTATTCATTATGTG
TTTGGCGGCGGCACGAAGTTAACCGTTCTTGGCCAGTGA

Additional antibodies generated from PRO-007 by point mutations also demonstrated high binding affinity to both FGFR2 and FGFR3. For example, antibody IB1, the V_{H} of which is presented hereafter in SEQ ID NO: 7 (generated by three point mutations in the V_{H} of PRO-007), binds both FGFR2 and FGFR3 with high affinity similar to PRO-007.

SEQ ID NO: 7:
VQLQQSGPGLVKPSQTLSLTCAISGDSVSSNSAAWSWIRQSPGRGLEWLGR
TYYRSKWYNDYAVSVKSRITINPDTSKNQFSLQLNGVTPEDTAVYYCARSY
YPDFDXWGQGTLVTV

PEGylation is a process of attaching one or more chains of polyethylene glycol (PEG) to a protein molecule. This process is intended to lengthen the life time of a substance in the bloodstream (without being metabolized and excreted by the body). The term “antibody” also includes modified forms of an antibody or its fragments, i.e. PEGylated scFv or an antibody (or an antibody fragment), optionally conjugated to a toxin molecule.

A “molecule having the antigen-binding portion of an antibody” as used herein is intended to include not only intact immunoglobulin molecules of any isotype and generated by any animal cell line or microorganism, but also the antigen-binding reactive fraction thereof, including, but not limited to, the Fab fragment, the Fab′ fragment, the F(ab′)₂ fragment, the variable portion of the heavy and/or light chains thereof, Fab mini-antibodies, dimeric bispecific miniantibodies, and chimeric or single-chain antibodies incorporating such reactive fraction, as well as any other type of molecule in which such antibody reactive fraction has been physically inserted, such as a chimeric T-cell receptor, or molecules developed to deliver therapeutic moieties by means of a portion of the molecule containing such a reactive fraction. Such molecules may be provided by any known technique, including, but not limited to, enzymatic cleavage, peptide synthesis or recombinant techniques.

The term “Fc” as used herein refers to the constant portion of an immunoglobulin molecule (Fragment crystallizable) that mediates phagocytosis, triggers inflammation and targets Ig to particular tissues; the Fc portion is also important in complement activation.

The “extracellular domain” when used herein refers to the polypeptide sequence of the FGFR2 which is normally positioned to the outside of the cell. The extracellular domain encompasses polypeptide sequences in which part of or all of the adjacent (C-terminal) hydrophobic transmembrane and intracellular sequences of the mature FGFR2 have been deleted. Thus, the extracellular domain-containing polypeptide can comprise the extracellular domain and a part of the transmembrane domain. According to some embodiments, the polypeptide comprises only the extracellular domain of the FGFR2. The truncated extracellular domain is generally soluble.

The term “epitope” is meant to refer to that portion of any molecule capable of being bound by an antibody or a fragment thereof which can also be recognized by that antibody. Epitopes or antigenic determinants usually consist of chemically active surface groupings of molecules such as amino acids or sugar side chains and have specific three-dimensional structural characteristics as well as specific charge characteristics.

An “antigen” is a molecule or a portion of a molecule capable of being bound by an antibody. An antigen may have one or more than one epitope. The specific reaction referred to above is meant to indicate that the antigen will react, in a highly selective manner, with its corresponding antibody and not with the multitude of other antibodies which may be evoked by other antigens.

A “neutralizing antibody” as used herein refers to a molecule having an antigen-binding site to a specific receptor capable of reducing or inhibiting (blocking) activity or signaling through a receptor, as determined by in vivo or in vitro assays, as per the specification.

A “monoclonal antibody” or “mAb” is a substantially homogeneous population of antibodies to a specific antigen. mAbs may be obtained by methods known to those skilled in the art. The mAbs of the present invention may be of any immunoglobulin class including IgG, IgM, IgE, IgA, and any subclass thereof. A hybridoma producing a mAb may be cultivated in vitro or in vivo. High titers of mAbs can be obtained by in-vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

Chimeric antibodies are molecules, the different portions of which are derived from different animal species, such as those having a variable region derived from a murine mAb and a human immunoglobulin constant region. Antibodies which have variable region framework residues substantially from human antibody (termed an acceptor antibody) and complementarity determining regions substantially from a mouse antibody (termed a donor antibody) are also referred to as humanized antibodies. Chimeric antibodies are primarily used to reduce immunogenicity in application and to increase yields in production, for example, where murine mAbs have higher yields from hybridomas but higher immunogenicity in humans, such that human/murine chimeric mAbs are used. Chimeric antibodies and methods for their production are known in the art.

Besides the conventional method of raising antibodies in vivo, antibodies can be generated in vitro using phage display technology. In addition, the present invention provides molecules comprising at least the antigen-binding portion of an antibody which has affinity for FGFR2, optionally with cross-reactivity to other FGF receptors, e.g. FGFR3. These molecules include antibodies specific to FGFR2 and/or FGFR3, peptide analogs of such antibodies with binding affinity to the extracellular portion of FGFR2 and/or FGFR3, and peptidomimetics based on the structure of such peptides.

### Polynucleotides

The terms “nucleic acid” and “polynucleotides” as used herein are interchangeable and refer to molecules such as deoxyribonucleic acid (DNA), and, where appropriate, ribonucleic acid (RNA). Nucleic acids or polynucleotides should also be understood to include, as equivalents, analogs of either RNA or DNA made from nucleotide analogs, and, as applicable, single (sense or antisense) and double-stranded polynucleotides.

Within the scope of the present invention is a nucleic acid molecule encoding an antibody having affinity for FGFR2, optionally with cross-reactivity to other FGF receptors, which block receptor activation. The nucleic acid molecule contains a nucleotide sequence having at least 75% sequence identity, preferably about 90%, and more preferably about 95% identity to the nucleotide sequence set forth in any one of SEQ ID NO:3, SEQ ID NO:4 or SEQ ID NO:6. In the hypervariable regions of the heavy chain and light chain, the nucleic acid molecule contains a nucleotide sequence having at least 50% sequence identity, preferably about 70% and more preferably about 80% identity to the molecule set forth in SEQ ID NO:6.

The invention also provides nucleic acids that hybridize under high stringency conditions to polynucleotides set forth in any one of SEQ ID NO:3, SEQ ID NO:4 or SEQ ID NO:6. or the complement thereof.

### Pharmaceutical compositions

The present invention provides pharmaceutical compositions for use in treating, attenutating the progression of, or alleviating one of more symptoms of multiple sclerosis, the pharmaceutical compositions comprise as an active agent one or more molecules which comprise at least the antigen-binding portion of an antibody which has affinity for FGFR2, optionally with cross-reactivity to other FGF receptors, e.g., FGFR3, and a pharmaceutically acceptable carrier.

The term “carrier” refers to a diluent or vehicle with which the active agent is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

The pharmaceutical composition can further comprise a pharmaceutically acceptable excipient. Examples of excipients include, but are not limited to, an agent for adjustment of tonicity such as, for example, sodium chloride or dextrose; a buffering agent such as, for example, phosphate buffer, acetate buffer, Tris buffer or citrate buffer; an emulsifying agent such as a surfactant which can be a nonionic, anionic, cationic, zwitterionic, and amphoteric pharmaceutically acceptable surfactant useful in medicaments for human

The pharmaceutical compositions of the present invention can be formulated as a liquid, such as a solution, suspension, or emulsion, i.e., oil-in-water emulsion, water-in-oil emulsion, microemulsion or nanoemulsion. The pharmaceutical composition can be prepared in a dried form, such as a lyophilized powder, which can be reconstituted into a liquid solution, suspension, or emulsion before administration, for example, by parenteral administration such as by subcutaneous, intramuscular or intravenous administration. The pharmaceutical compositions are preferably sterilized by membrane filtration and are stored in unit-dose or multi-dose containers such as sealed vials or ampoules.

The pharmaceutical compositions can also take the form of tablets, capsules, sustained-release formulations and the like. The pharmaceutical composition can also be formulated as a suppository, with traditional binders and carriers such as triglycerides, microcrystalline cellulose, gum tragacanath or gelatin.

The dosage of the active agent of the present invention varies depending on the administration route, age, body weight, sex, or the condition of the patient. If the pharmaceutical composition is administered by parenteral administration, the daily dosage can generally be between about 0.001 mg to about 100 mg, preferably between about 0.001 mg to about 10 mg, more preferably between about 0.01 mg to about 1 mg, per kg body weight. If the pharmaceutical composition is administered orally, the daily dosage can generally be between about 0.01 mg to about 500 mg, preferably between about 0.01 mg to about 50 mg, more preferably between about 0.1 mg to about 10 mg, per kg body weight. Various considerations in arriving at an effective amount are described, e.g., in Goodman and Gilman's: The Pharmacological Bases of Therapeutics, 8th ed., Pergamon Press, 1990; and Remington's Pharmaceutical Sciences, 17th ed., Mack Publishing Co., Easton, Pa., 1990.

### Methods of Use

The present invention provides a method for treating, attenuating the progression of, or alleviating one or more symptoms of multiple sclerosis, the method comprising administering to a subject in need of such treatment a therapeutically effective amount of a pharmaceutical composition comprising as an active agent an antibody or an antigen biding fragment thereof having high binding affinity to FGFR2 and a pharmaceutically acceptable carrier.

The term “treating” as used herein includes, but is not limited to, any one or more of the following: abrogating, ameliorating, inhibiting, attenuating, blocking, suppressing, reducing, halting, alleviating or preventing symptoms associated with multiple sclerosis. Thus, treating a subject as used herein refers to any type of treatment that imparts a benefit to a subject afflicted with multiple sclerosis (MS), including improvement in the condition of the subject (e.g., in one or more symptoms), delay in the progression of the disease, delay the onset of one or more symptoms, or slow the progression of one or more symptoms.

The efficacy of the treatment may be measured in any manner. For example, treatment may result in an inhibition or lessening of one or more symptoms of the disease, may ameliorate the disease, or may cure the disease. For subjects with MS, this may be measured by reducing the recurrence of the disease in a subject, prolonging overall survival of a subject or prolonging the progression-free time period or disease-free time period.

Symptoms of multiple sclerosis include, but are not limited to, muscle weakness, difficulties in balance, impairment of speech, impairment in swallowing, vision loss, and fatigue.

A “therapeutically effective amount” as used herein means the amount of the active agent that is sufficient to effect a treatment (as defined above). The therapeutically effective amount will vary depending on the active agent, formulation, the severity of the disease, and the age, weight, physical condition and responsiveness of the subject to be treated.

The pharmaceutical compositions of the invention can be administered via a parenteral route of administration, such as by intravenous, intramuscular, subcutaneous, intraarterial and intraperitoneal injection or infusion. Alternatively, the pharmaceutical compositions can be administered topically, nasally, or by oral, rectal or vaginal administration route. Each possibility represents a separate embodiment of the invention. In an exemplary embodiment, the pharmaceutical composition is administered by injection.

Studies of the natural history of MS suggest that there are different patterns of disease activity. Some patients have rare attacks, some have frequent attacks, and others gradually but steadily worsen without experiencing attacks. Patients who have rare attacks and are minimally disabled ten years after being diagnosed with MS are said to have benign MS. This group constitutes only about 10-15% of the total MS patient population, although there is some evidence suggesting that this course may be more common than is currently appreciated. Patients who have attacks with full or partial recovery and are otherwise stable between attacks are defined as having relapsing-remitting MS. Approximately 80-90% of patients with MS initially experience a relapsing-remitting course. Of these, approximately 50% will have difficulty walking 15 years after onset and 80% will ultimately (after about 25 years) experience gradual progression of disability with or without attacks. Patients who first experience exacerbations and later experience gradual progression of disability have secondary progressive MS. Approximately 10-15% of MS patients do not experience an initial attack. Those patients who gradually worsen after the appearance of the first symptom have primary progressive MS. A few patients with primary progressive MS will later experience an exacerbation. These patients have progressive-relapsing MS.

There is as yet no cure for MS. Many medications of MS have serious side effects and some carry significant risks. However, three forms of beta interferon (AVONEX® (interferon beta-1a), BETASERON® (interferon beta-1b), and REBIF® (interferon beta-1a)) have been approved by the Food and Drug Administration (FDA) for treatment of relapsing-remitting MS. Beta interferon has been shown to reduce the number of exacerbations and may slow the progression of physical disability. When attacks do occur, they tend to be shorter and less severe. The FDA also has approved a synthetic form of myelin basic protein copolymer I, COPAXONE® (glatiramer acetate), for the treatment of relapsing-remitting MS. Copolymer I has few side effects, and studies indicate that the agent can reduce the relapse rate by almost one third. An immunosuppressant treatment, NOVATRONE® (mitoxantrone) is also approved by the FDA for the treatment of advanced or chronic MS.

The humanized antibody Ocrelizumab (Ocrevus) is approved by the FDA to treat both relapse-remitting MS and primary progressive MS.

According to some embodiments, the present invention provide a combination therapy which comprises a step of administering a first pharmaceutical composition comprising the antibody or an antigen-binding fragment thereof having high binding affinity to FGFR2, optionally with cross-reactivity to other FGFR2, e.g., FGFR3, the method further comprises a step of administering a second pharmaceutical composition comprising a therapeutic agent known to treat MS.

The therapeutic agents known to treat MS include, but not limited to, ocrelizumab (Ocrevus™), beta interferons, glatiramer acetate (Copaxone®), dimethyl fumarate (Tecfidera®), fingolimod (Gilenya™), teriflunomide (Aubagio), natalizumab (Tysabri®), and alemtuzumab (Lemtrada®).

The composition comprising the antibody and the composition comprising a therapeutic agent known to treat MS may be administered as two separate dosage forms via the same or different routes. For example, the composition comprising the antibody and the composition comprising a therapeutic agent known to treat MS may be administered such that one is administered before the other with a difference in administration time of 5 minutes, 10 minutes, 30 minutes, 1 hour, 2 hours, 4 hours or more or any integer in between.

According to one embodiment, the first pharmaceutical composition comprising the antibody, and the second pharmaceutical composition comprising a therapeutic agent known to treat MS are administered concurrently. According to another embodiment, said first and second pharmaceutical compositions are administered at different regimens. Examples of regimens for injections include, but are not limited to, once daily for one, two, three, four, five, six, seven or more consecutive days, three times a week, twice a week, every other day, once a week, once in two weeks, once in three weeks, once a month, etc.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### EXAMPLE 1

### Effect of Anti-FGFR2/3 Antibodies on EAE Manifestations

Experimental autoimmune encephalomyelitis (EAE) is the most common animal model for multiple sclerosis (MS) sharing many clinical and pathophysiological features. Actively-induced EAE in mice is the easiest inducible model with robust and replicable results. C57BL/6 mice are the commonly used strain. The mice are immunized with myelin oligodendrocyte glycoprotein (MOG) 35-55 peptide, and within 9-14 days after immunization, the mice develop EAE with paralysis (see, for example, Bittner et al., 2014, J. Vis. Exper. (86) doi: 10.3791/51275).

Four groups (10 female mice/group) were immunized with MOG 35-55 peptide. After the onset of the disease, each group was treated as indicated in Table 1 below.

TABLE 1
Group designation.

| | | | Treatment |
|---|---|---|---|
| Group ID | Material | Dose (mg/kg) | frequency |
| A | PRO-007 | 0.08 | 48 hrs |
| B | PRO-007 | 0.08 | 24 hrs |
| C | PRO-007 | 1.2 | 48 hrs |
| D | PBS | N/A | 48 hrs |

PRO-007 is a single chain antibody fragment which binds both FGFR2 and FGFR3 with high affinity and has the amino acid sequence of SEQ ID NO:5 as follows:

PRO-007 SINGLE CHAIN
(SEQ ID NO: 5)
MVQLQQSGPGLVKPSQTLSLTCAISGDSVSSNSAAWNWIRQSPGRGLEWLG
RTYYRSKWYNDYAVSVKSRITINPDTSKNQFSLQLNSVTPEDTAVYYCARS
YYPDFDYWGQGTLVTVSSAGGGSGGGGSGGGGSGGGGSDIELTQPPSVSVA
PGQTARISCSGDALGDKYASWYQQKPGQAPVLVIYDDSDRPSGIPERFSGS
NSGNTATLTISGTQAEDEADYYCQSYASQGIHYVFGGGTKLTVLGQ

After the onset of the disease, treatment was conducted by intraperitoneal (ip) injection of PRO-007 (100 μl of 1 mg/ml) every 24 hrs or 48 hrs at the indicated amounts (see Table 1) for a total treatment period of 21 days.

Clinical score and animal body weight were measures daily. At the end of the study (after 45 days), mice were sacrificed by CO₂, and the spinal cord and brain were kept in paraformaldehyde (PFA; 4%). Samples were then evaluated for histological demyelination using luxor blue staining.

PRO-007 was subjected to endotoxin removal by EndoSafe® cartridge, and Limulus amebocyte lysate (LAL) concentration was determined to be 270 EU/ml or 270 EU/mg.

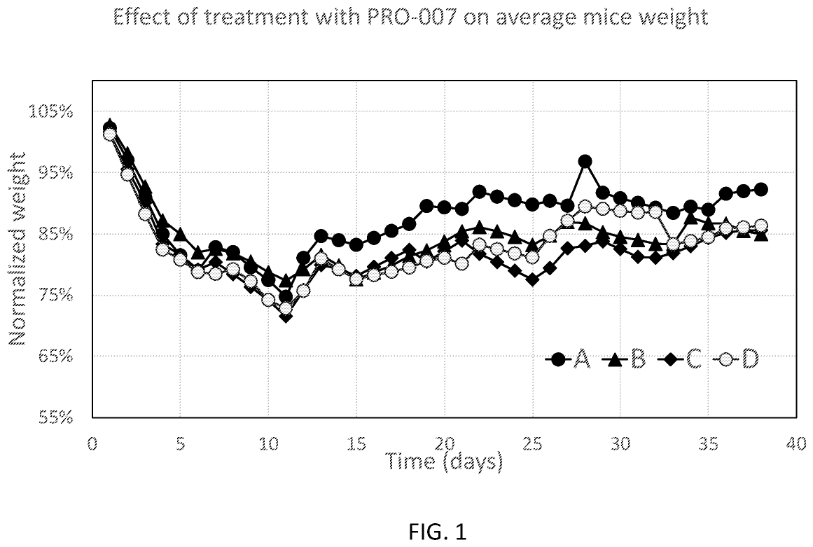

FIG. 1 shows the effect of PRO-007 on body weight of the EAE mice. As seen in FIG. 1, the body weight of the PRO-007 treated mice was essentially similar to that of the control mice.

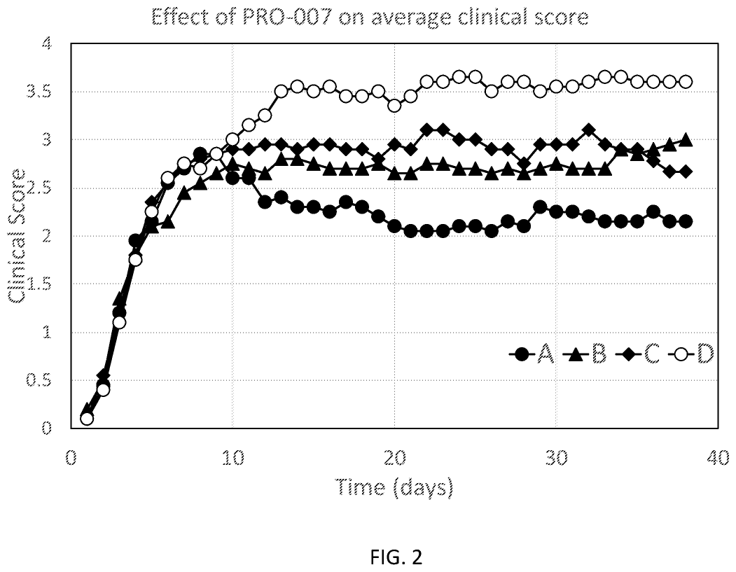

FIG. 2 shows the clinical scoring in EAE mice. As seen in FIG. 2, the clinical scoring of the untreated EAE mice was on average about 3.5 (Group D; FIG. 2). The clinical scoring of the EAE mice injected with anti-FGFR2/3 antibodies PRO-007 at a dose of 0.08 mg/kg every 48 hrs was on average about 2.2 (Group A; FIG. 2). Injection of the antibodies PRO-007 at a dose of 0.08 mg/kg every 24 hrs (Group B; FIG. 2) or injection of the antibodies PRO-007 at a dose of 1.2 mg/kg every 48 hrs (Group C; FIG. 2) resulted in some improvement in the clinical scoring as compared to the untreated mice, but this improvement was lower than that observed in Group A mice.

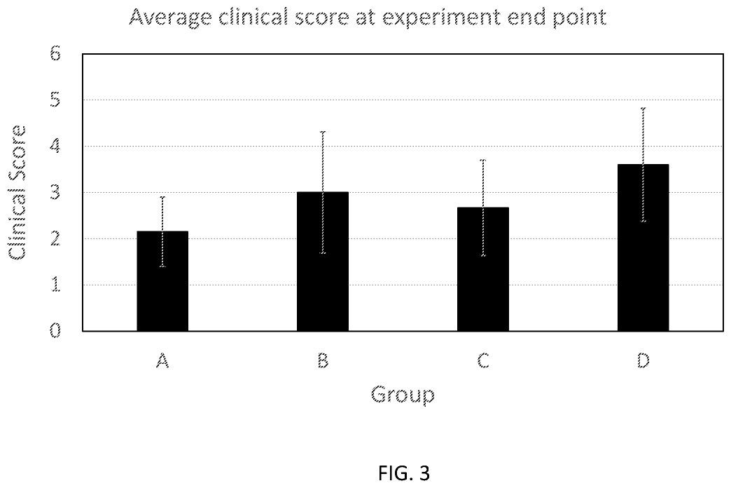

The clinical scoring of the EAE mice at the end of the experiment is shown in FIG. 3. A statistical significant difference was observed between the clinical scoring of the untreated mice (Group D; FIG. 3) and that of the mice treated the anti-FGFR2/3 antibodies PRO-007 at a dose of 0.08 mg/kg every 48 hrs (Group A; FIG. 3).

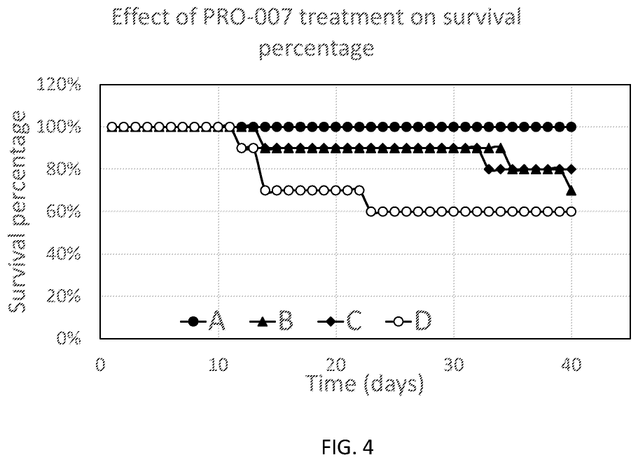

The survival rate of the EAE mice is shown in FIG. 4. While untreated EAE mice showed 60% survival at the end of the experiment (Group D; FIG. 4), mice treated with PRO-007 at a dose of 0.08 mg/kg every 48 hrs showed 100% survival (Group A; FIG. 4). Mice treated with PRO-007 at a dose of 1.2 mg/kg every 48 hrs or mice treated with PRO-007 at a dose of 0.08 mg/kg every 24 hrs showed higher survival rate than the untreated mice, but lower survival rate than the mice treated with PRO-007 at a dose of 0.08 mg/kg every 48 hrs.

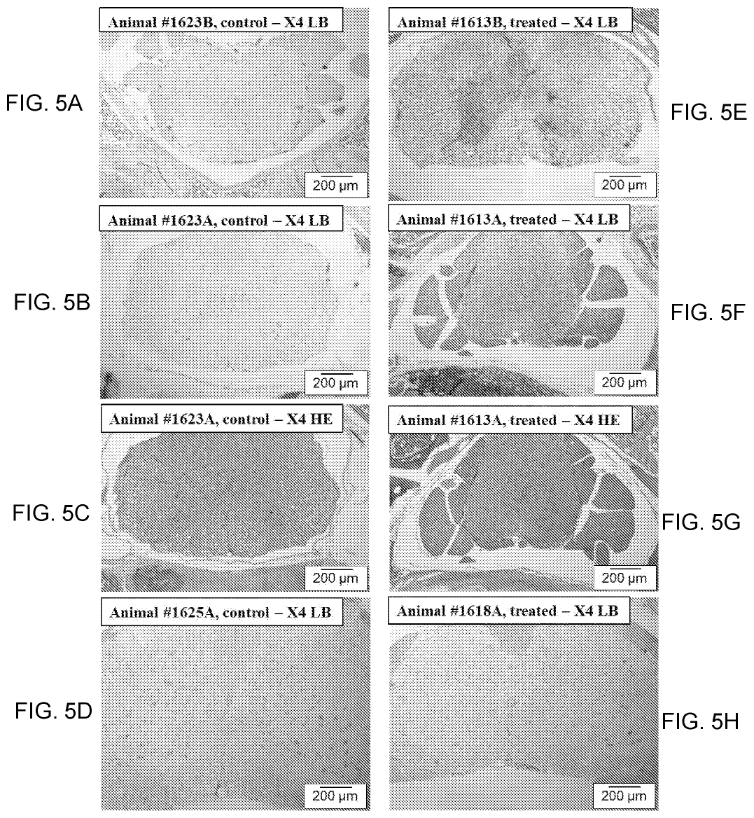

Brain sections from untreated EAE mice and from EAE mice treated with PRO-007 at a dose of 0.08 mg/kg every 48 hrs were stained with luxor blue and are shown in FIG. 5. As shown in FIG. 5, brain sections from untreated mice showed areas of high demyelination, while brain sections from EAE mice treated with PRO-007 (Group A) showed limited demyelination.

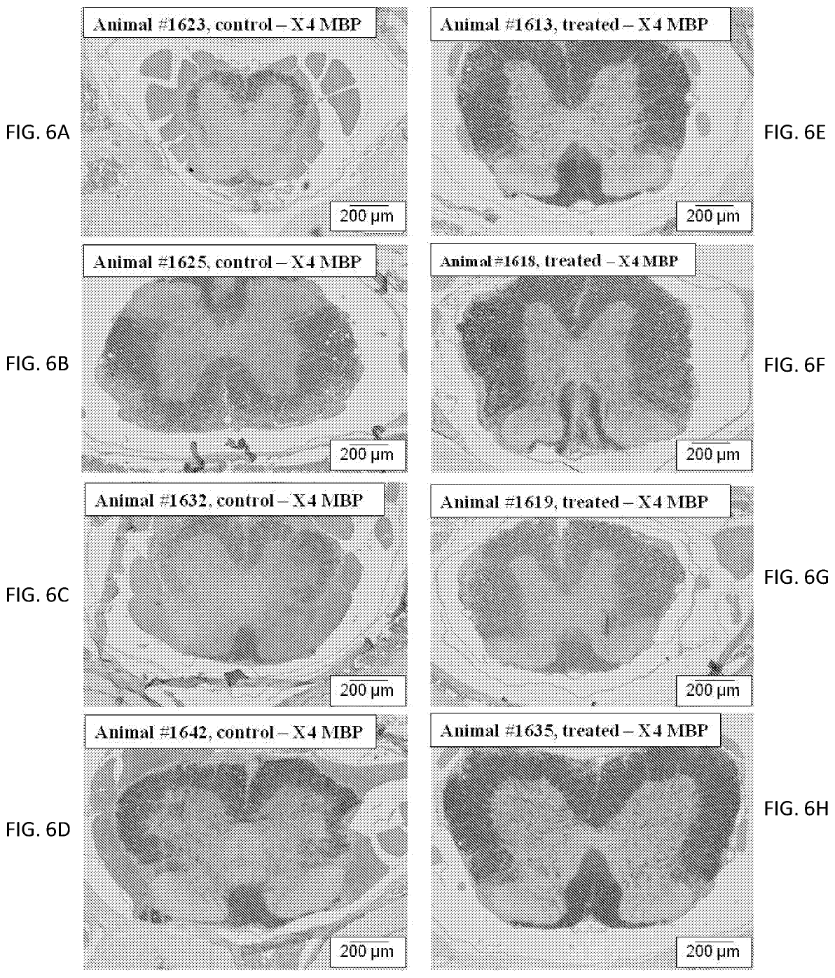

Immunohistochemical analysis of myelin basic protein (MBP) in brain sections indicated that EAE mice treated with the antibodies PRO-007 (Group A) showed larger areas of healthy white matter than untreated EAE mice (FIG. 6).

In order to quantitate the area of the healthy white matter tissue, the following calculation was performed:

(T−GM−L)/(T−GM)
Where—
T is the total area of slice
GM is the area of gray matter (T−GM=White matter area)
L is the area of lesions (T−GM−L=healthy white matter area)

TABLE 1
A summary of the immunohistochistry evaluation
of the healthy white matter.

| Animal # | Group | MBP grade | Additional changes |
|---|---|---|---|
| 1623A | Control | 1 | non |
| 1623B | Control | 2 | non |
| 1623C | Control | 2 | non |
| 1623D | Control | 2 | non |
| 1623E | Control | Artefact | non |
| 1625A | Control | 2 | non |
| 1625B | Control | 2 | non |
| 1625C | Control | 3 | non |
| 1625D | Control | 1 | non |
| 1625E | Control | Artefact | non |
| 1632A | Control | 3 | non |
| 1632B | Control | 2 | non |
| 1632C | Control | 1 | non |
| 1632D | Control | 2 | non |
| 1632E | Control | Artefact | non |
| 1642A | Control | 2 | non |
| 1642B | Control | 2 | non |
| 1642C | Control | 1 | non |
| 1642D | Control | 3 | non |
| 1642E | Control | Artefact | non |
| | Average | 1.93 | |
| 1613A | Treated | 4 | non |
| 1613B | Treated | 5 | non |
| 1613C | Treated | 4 | non |
| 1613D | Treated | Artefact | non |
| 1613E | Treated | Artefact | non |
| 1618A | Treated | 4 | non |
| 1618B | Treated | 3 | non |
| 1618C | Treated | 5 | non |
| 1618D | Treated | 2 | non |
| 1618E | Treated | Artefact | non |
| 1619A | Treated | 4 | non |
| 1619B | Treated | 4 | non |
| 1619C | Treated | 2 | non |
| 1619D | Treated | 3 | non |
| 1619E | Treated | Artefact | non |
| 1635A | Treated | 5 | non |
| 1635B | Treated | 4 | non |
| 1635C | Treated | 3 | non |
| 1635D | Treated | 2 | non |
| 1635E | Treated | Artefact | non |
| | Average | 3.6 | |

As shown in Table 1, the area of the healthy white matter was larger in brains of EAE mice treated with PRO-007 as compared to untreated EAE mice.

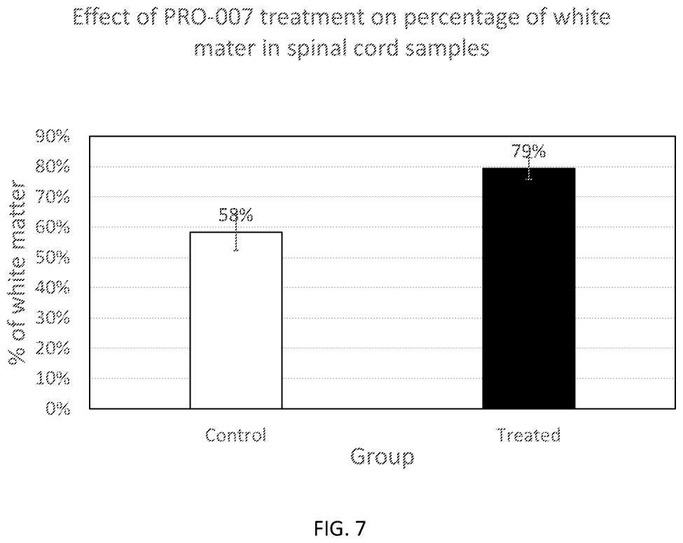

Evaluation of the healthy white matter in spinal cord samples from EAE untreated mice and EAE mice treated with the antibodies PRO-007 is shown in FIG. 7. As shown in FIG. 7, the % of healthy white matter in spinal cord samples of EAE mice treated with PRO-007 was higher than that of EAE untreated mice.

These results indicate that treatment of EAE mice with anti-FGFR2/3 antibodies is effective in reducing the disease manifestations, i.e., paralysis, mortality, and demyelination.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-17. (canceled)

18. A method of treating, slowing the progression of, or ameliorating one or more symptoms of multiple sclerosis, the method comprising administering to a subject in need of such treatment, a pharmaceutical composition comprising a therapeutically effective amount of an antibody or an antigen-binding portion thereof having affinity to fibroblast growth factor receptor 2 (FGFR2), optionally with cross-reactivity to another FGFR.

19. The method according to claim 18, wherein the antibody or the antigen-binding portion thereof has affinity to both FGFR2 and FGFR3.

20. The method according to claim 19, wherein the antibody or the antigen-binding portion thereof binds both FGFR2 and FGFR3 with an affinity of at least 50 nM.

21. The method according to claim 20, wherein the antibody or the antigen-binding portion thereof binds both FGFR2 and FGFR3 with an affinity of at least 10 nM.

22. The method according to claim 18, wherein the antibody or the antigen-binding portion thereof is substantially devoid of affinity to FGFR1.

23. The method according to claim 18, wherein the antibody or the antigen-binding portion thereof is selected from the group consisting of a monoclonal antibody, a proteolytic fragment of the monoclonal antibody, and a single chain antibody.

24. The method according to claim 18, wherein the antibody or the antigen-binding portion thereof comprises a V_{H}-CDR3 region having an amino acid sequence as set forth in SEQ ID NO:1 and a V_{L}-CDR3 region having an amino acid sequence as set forth in SEQ ID NO:2.

25. The method according to claim 23, wherein the proteolytic fragment of the antibody is a single chain Fv.

26. The method according to claim 25, wherein the single chain Fv has an amino acid sequence as set forth in SEQ ID NO:5.

27. The method according to claim 18, wherein administering the pharmaceutical composition is performed by intravenous, intraarterial, intramuscular, or intraperitoneal injection or infusion.

28. The method according to claim 27, wherein administering the pharmaceutical composition is performed by intravenous, intraarterial, intramuscular, or intraperitoneal injection or infusion once daily for 1, 2, 3, 4, 5, 6, or more consecutive days.

29. The method according to claim 27, wherein administering the pharmaceutical composition is performed by intravenous, intraarterial, intramuscular, or intraperitoneal injection or infusion every other day for 2, 3, 4, 5, or more times.

30. The method according to claim 18, further comprising a step of administering a pharmaceutical composition comprising a therapeutically effective amount of an agent known to treat multiple sclerosis.

31. The method according to claim 30, wherein the agent known to treat multiple sclerosis is selected from the group consisting of ocrelizumab, beta interferon, glatiramer acetate, dimethyl fumarate, and fingolimod.

32. The method according to claim 30, wherein administering the pharmaceutical composition is performed by oral or parenteral administration route.

33. The method according to claim 32, wherein the parenteral administration route is selected from the group consisting of intravenous, intraarterial, intramuscular, and intraperitoneal injection or infusion.

34. The method according to claim 30, wherein administering the pharmaceutical compositions comprising the antibody or antigen-binding portion thereof having affinity to both FGFR2 and FGFR3 and the pharmaceutical composition comprising the agent known to treat multiple sclerosis is performed concomitantly or subsequently to each other.
